(19) United States
(12) Patent Application Publication
(10) Pub. No.: US 2016/0206871 A1
(43) Pub. Date: Jul. 21, 2016

(54) VARIABLY CONFIGURABLE, ADAPTABLE ELECTRODE ARRAYS AND EFFECTUATING SOFTWARE, METHODS, AND SYSTEMS

(71) Applicant: Rio Grande Neurosciences, Inc., Santa Fe, NM (US)

(72) Inventor: Michael Weisend, Yellow Springs, OH (US)

(73) Assignee: Rio Grande Neurosciences, Inc., Santa Fe, NM (US)

(21) Appl. No.: 14/083,379

(22) Filed: Nov. 18, 2013

Related U.S. Application Data
(60) Provisional application No. 61/796,634, filed on Nov. 16, 2012.

(51) Int. Cl. A61N 1/04; A61N 1/36

(52) U.S. Cl. CPC A61N 1/0476; A61N 1/0456; A61N 1/36025

(57) ABSTRACT

Electrical non-invasive brain stimulation (NIBS) delivers weak electrical currents to the brain via electrodes that are affixed to the scalp. NIBS can excite or inhibit the brain in areas that are impacted by that electrical current during and for a short time following stimulation. Electrical NIBS can be used to change brain structure in terms of increasing white matter integrity as measured by diffusion tensor imaging. Together the electrical NIBS can induce changes in brain structure and function. The present methods and devices are adaptable to and configurable for facilitating the enhancement of brain performance, and the treatment of neurological diseases and tissues. The present methods and devices are advantageously designed to utilize modern electrodes deployed with, inter alia, various spatial arrangements, polarities, and current strengths to target brain areas or networks to thereby enhance performance or deliver therapeutic interventions.

### RELATED APPLICATIONS AND PRIORITY

This Utility Patent Application claims the priority and benefit of commonly owned U.S. Provisional Patent Application Ser. No. 61/796,634 of Michael P. Weisend, as filed 16 Nov. 2012, entitled “Systems, Methods, Software And Devices For The Non-Invasive Testing Evaluation And Stimulation Of Portions Of The Brain To Enhance Learning And Communications Skills, Or To Treat Neurological Disorders.” This Utility Patent Application also claims the priority and benefit of commonly owned U.S. Provisional Patent Application Ser. No. 61/962,698 of Michael P. Weisend, as filed 14 Nov. 2013, entitled “Variably Configurable, Adaptable Electrode Arrays And Effectuating Software, Methods And Systems For Determining The Maximum Operational Parameters Of External Neurological Electrodes And Electrode Arrays, And Systems And Methods For Identifying Brain Regions Most Amenable To Non-Invasive Brain Stimulation, All Adaptable For The Non-Invasive Testing, Evaluation And Stimulation Of Portions Of The Brain To Enhance Learning And Communications Skill s, Or To Treat Neurological Disorders.” Both of these provisional patent applications are hereby incorporated by reference in their entireties into the present patent application. Also hereby incorporated by reference in their entireties are each of the references cited in the present application, as well as those cited in Provisional Patent Application Ser. Nos. 61/796,634, and 61/962,698 as identified herein.

### THE TECHNOLOGY

The present technology relates to the use of various electrical means, variably configurable and adaptable electrodes and electrode arrays, methods and software for affecting electrical communication systems that naturally occur in the brain, and for effecting various kinds of advantageous neurological intervention, such as redirecting learning processes. The various types and intensities of electrical stimulation may be adapted and arranged to amplify, or to cancel, targeted portions and functions of the brain. The technology has numerous uses, applications and embodiments.

### BACKGROUND

The application of electric fields or stimuli to the brain has been demonstrated for a variety of neurological conditions, including the treatment of psychological disorders. Attempts have been made to utilize such electrical stimuli to aid in the learning and teaching processes. However, many of the known methods involve invasive surgical procedures that carry considerable risk. While some non-invasive methodologies have begun to show promise, novel devices, systems, networks and methods are needed to address a variety of conditions and circumstances for which electrotherapies can be helpful. This is especially true in the teaching and scholarly fields, particularly as applied to personnel in the therapeutic, intelligence and military education fields.

### BRIEF DESCRIPTION OF THE DRAWINGS

FIG. 1 shows a headgear form electrical NIBS.

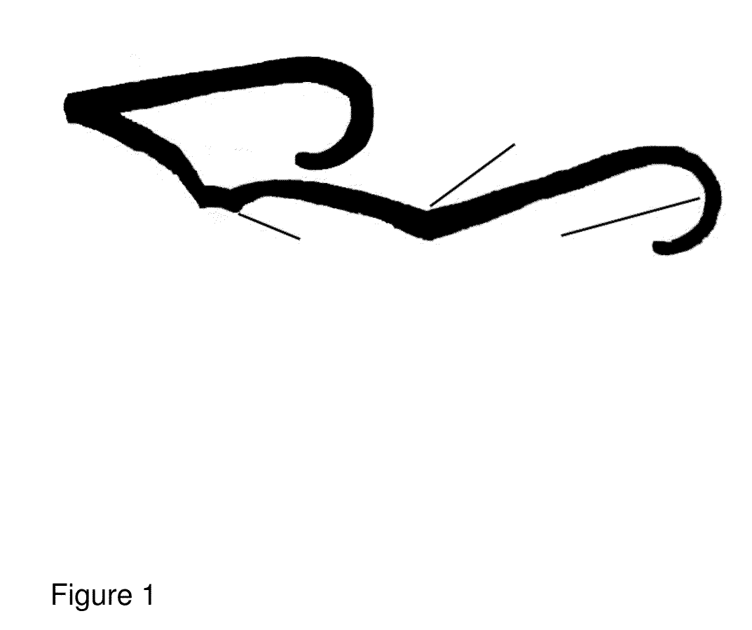

FIG. 2 shows an empty case for storage of headgear, electrodes, electronics and battery.

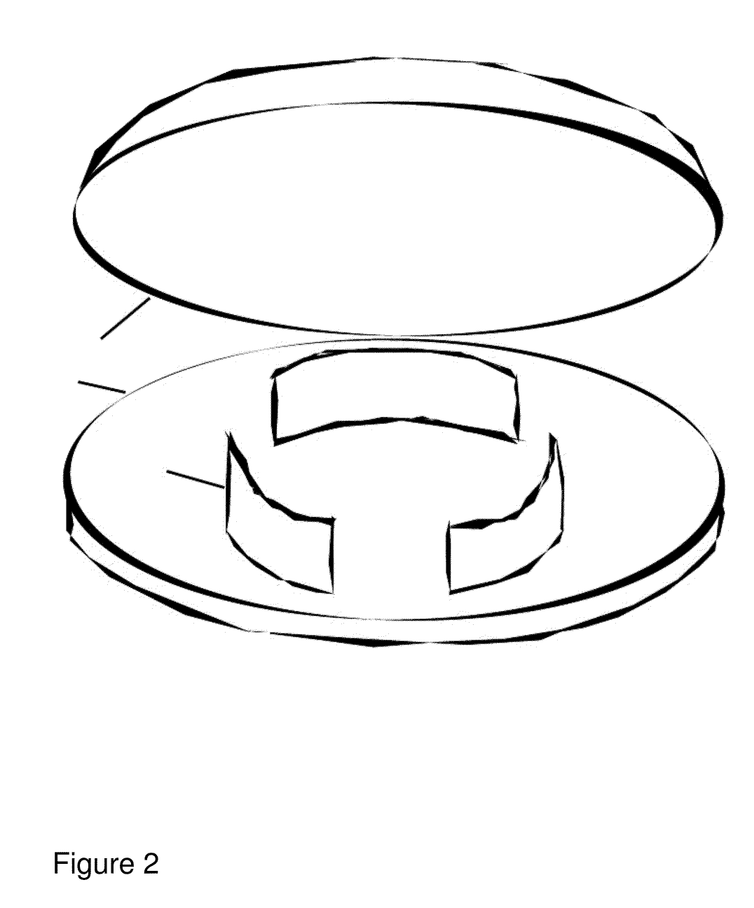

FIGS. 3A and 3B show an external view of case housing.

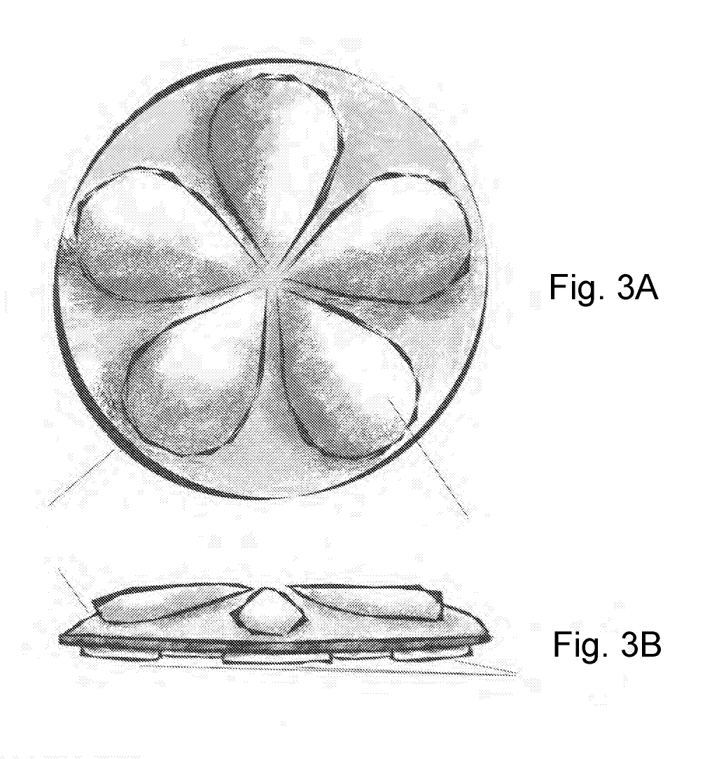

FIG. 4 shows folded head gear.

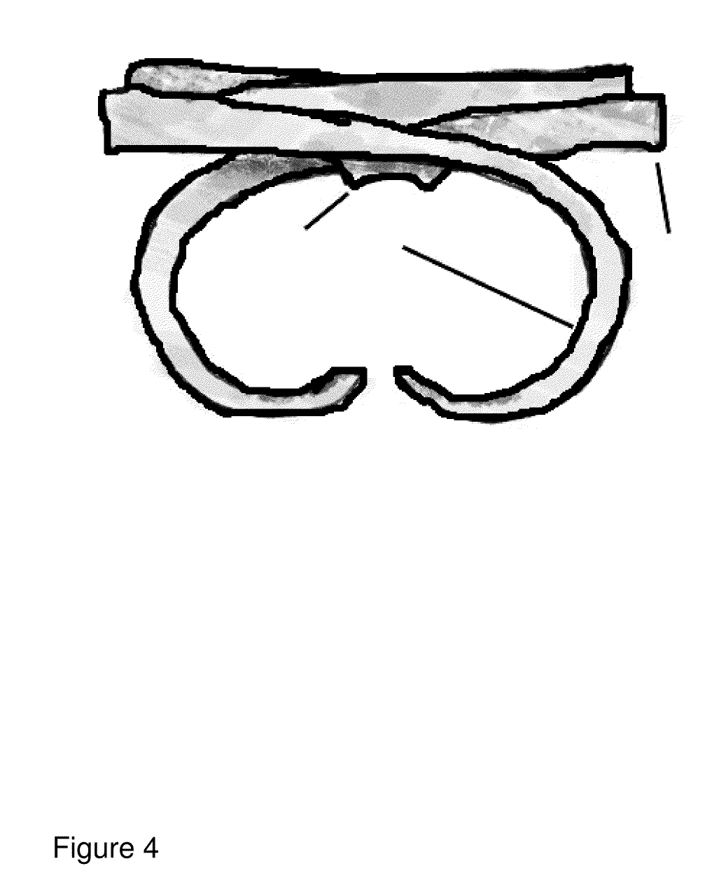

FIG. 5 shows a person wearing a possible headgear configuration.

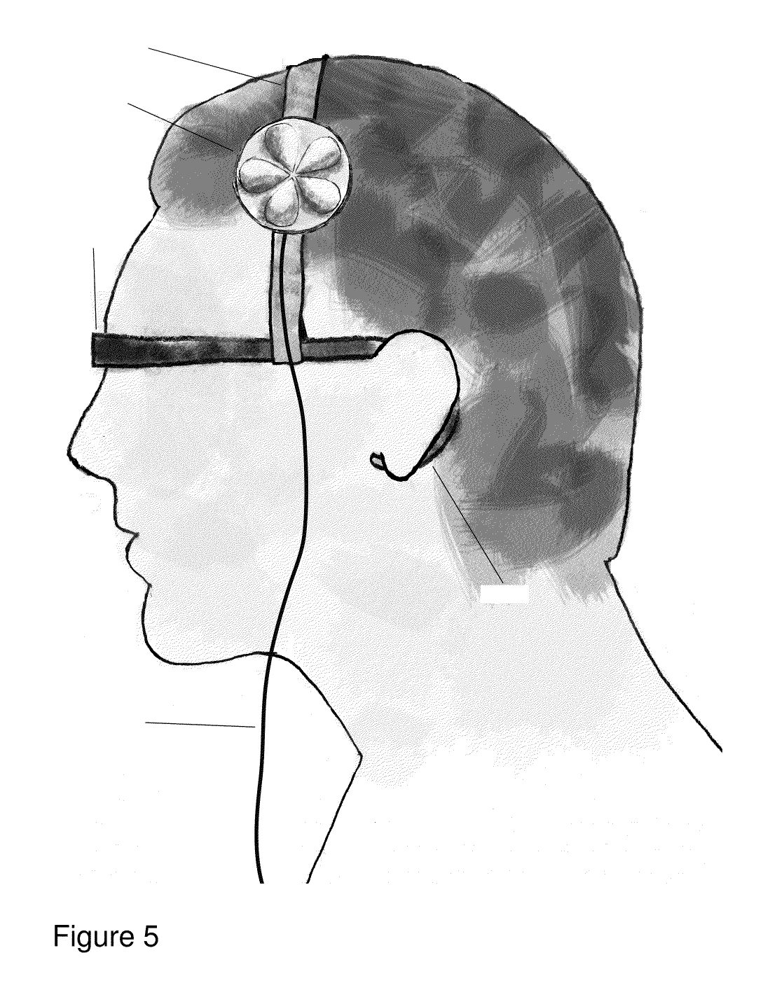

FIG. 6 shows a filled electrical NIBS device housing.

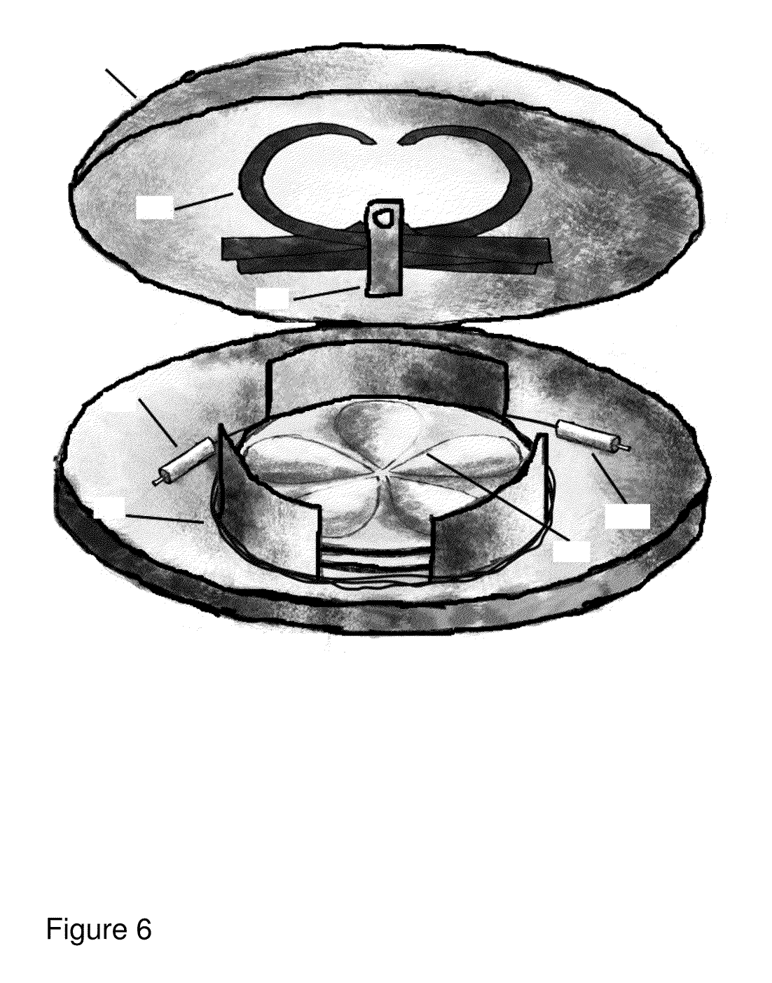

FIG. 7 shows a view of an electrode array.

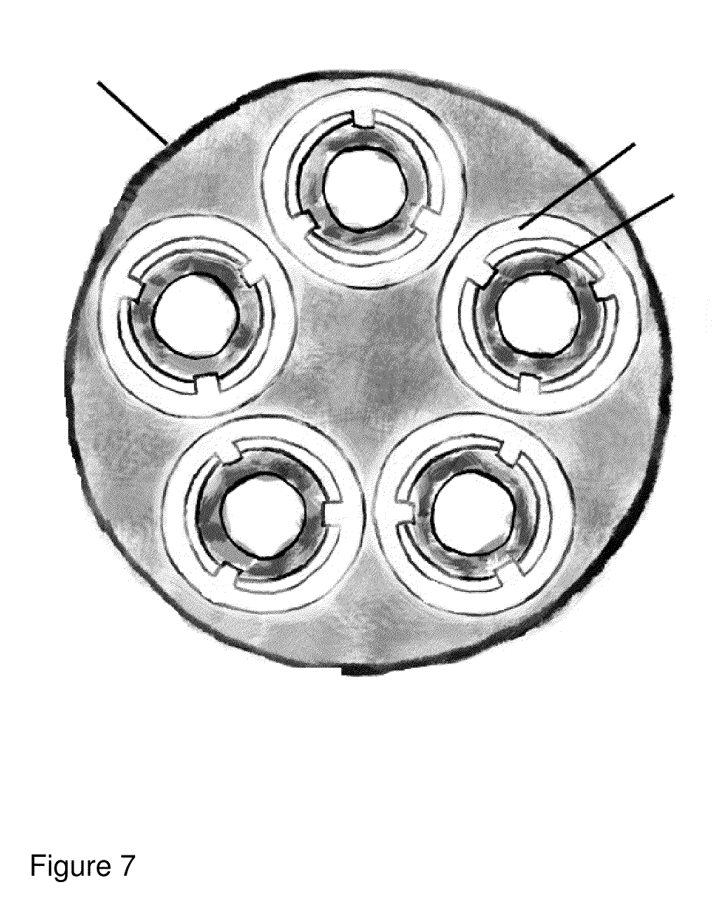

### DESCRIPTION OF THE INVENTION

According to the many and various embodiments of the present technology, the present disclosure provides, inter alia, various devices, methods, networks and systems for providing therapeutic and/or beneficial cranial electro-stimulation. Accordingly, the present disclosure provides methods, systems, software and apparatus that utilize a combination of real time brain functional monitoring and non invasive electrical and/or magnetic trans-cranial brain stimulation to modify the brain function as exhibited in individual and group activities.

The present technology includes several methods and devices whereby neurological interventions are effected essentially by electrical NIBS-based procedures accomplished by the devices and methods described herein. In accordance with the present technology, these methods and devices are provided to be directed toward specific tasks, diseases, disorders and treatments. In one aspect, these devices and methods are adapted and arranged to be used on one or a plurality of subject brains for various purposes, affects and results.

In another aspect one embodiment of a method of the invention includes the step or act ion of 1) recording brain activity (data) by means of one or more of magnetoencephalography (MEG), electroencephalography (EEG), functional magnetic resonance imaging (fMRI), positron emission tomography (PET), single photon emission computed tomography (SPECT), electrocorticography (ECOG), structural magnetic resonance imaging (sMRI), diffusion tensor imaging (OTT), magnetic resonance spectroscopy (MRS), and functional near infrared spectroscopy (fNIRS) during desirable and non-desirable brain states. Examples of desirable brain states which are useful for practicing the present methods include: attentive, expert, healthy, uninjured, cognitively fast, and cognitively flexible. Examples of undesirable brain states include: inattentive, untrained, depressed, brain injured (such as TBI), cognitively slow, cognitively rigid.

In another aspect, one embodiment of a method of the invention includes the step or action of 2) evaluating the differences in the subject brain or brains between desirable and non desirable brain states. This difference evaluation is performed with respect to the brain activity data obtained by one or more of the initial steps or actions of this embodiment of the method of the invention. The results of this difference evaluation between desirable and non desirable brain states can then be used to determine portions, regions or parts of the subject brain or brains which are suitable targets for electrical NIBS. By effecting NIBS of these target parts of the subject brain or brains, brain circuitry can influenced to transition from an undesirable to a desirable state. The advantages of this transition can be numerous.

In yet another important aspect of the present technology, the data obtained in the present method can be used to 3) effect a determination of one or more advantageous electrode array designs and configurations which are suitable for specific desired purposes, such as the teaching of languages, the enhancing of decision making, the increasing of vigilance, the increasing of cognitive flexibility, the enhancing of creativity, the teaching of the correct accents for languages, the increasing of attention, the enhancing of sleep the reversing of brain damage (such as that associated with traumatic brain injury, stroke, concussion, hypoxia, and chemical or other injury), and the treating of symptoms of mental illness (i.e. reducing hallucinations, elevating mood, alleviating flattened affect, reducing anxiety, reducing insomnia, reducing unwanted memory, enhancing social skills, reducing repetitive thoughts, reducing social phobias). With the present technology, electrodes and electrode arrays can be designed and configurations of arrays, including how the electrodes communicate with one another and with other components of the invention, can be effected to 3) maximize the effectiveness of NIBS-based neurological interventions. Such designs and configurations can be effected with respect to, among other factors, spatial positions of the electrodes in two or more dimensions, the respective polarities of the electrodes, the timing of activity on or between electrodes, the frequency (typically in terms of Hz) delivered by means of one or more electrodes, the frequency of stimulation in terms of repetition of a determined stimulation regimen, the latency of the stimulation, if any, on an electrode or electrodes with respect to environmental events, the correlation among stimulation parameters across electrodes, the correlation of stimulation with environmental events, the phase relationships among stimulation parameters across electrodes, the duration of stimulation on an electrode or electrodes and the relationships of these durations across electrodes, causal inferences from recordings of activity that are replayed to the brain via an electrode or electrodes, and the intensity or intensities of the electrical stimulation delivered by the respective electrodes, such that the NIBS stimulations can have the greatest desirable influence on targeted brain areas.

In a separate aspect, such designing and configuring of the individual electrodes and arrays of electrodes can be accomplished further by applying finite element modeling to data gathered regarding the brain, scalp, skull and associated tissues. In this aspect, the finite element model can be adapted and arranged to function as a filter with respect to the influence of the electrical NIBS on one or more portions, regions or areas of one or more target brains. With the gathered data, and with the assistance of finite element analysis, the relative configuration(s) and design(s) of the electrodes and electrode arrays can be effected with respect to various brain tissues in terms of, for example, the spatial distribution, polarity, and intensity of the excitation or inhibition delivered via NIBS. The combination of these systems, methods, devices, components and elements of the present technology are directed toward an efficient and effective step or activity of 4) stimulating one or more target brains with electrical NIBS. As another advantage, one or more of the polarity, intensity, and spatial distribution of stimulation can be programmed into the devices and arrays described herein to produce maximal influence (excitation or inhibition) at the target sites.

In yet another positive and innovative aspect, the present technology includes many embodiments of the recordation of multiple brain states: The present REDS technique takes advantage of the fact that there are desirable brain states that lead to behaviors that are well suited to the tasks and undesirable brain states that lead to poor performance on the same tasks. Desired brain states that aide performance could be attentive, happy, expert, quick while comparable undesirable brain states might be inattentive, sad, untrained, or slow, injured. REDS uses data from MEG, EEG, fMRI, PET, SPECT, ECOG, fNERS, sMRI, DTI, MRS, and other technologies to record data in the desirable and undesirable brain states, and in one embodiment, maps the recorded brain activity to the structures of origin using commonly available algorithms. The mapping to brain structures is done twice, once for desirable and again for undesirable brain states. This provides the basis for comparing and contrasting the structural and functional brain states that contribute to the difference between performance with desirable and undesirable outcomes. Thereby, determining the target brain region(s) where the influence of electrical NTBS could move the user from an undesired to a desired brain state. The REDS approach differs from the standard practice of electrical NIBS where neuroimaging is rarely used determine target brain structures. When no neuroimaging is performed the user must rely on often poorly founded assumptions about the electrical NIBS and the brain.

For evaluating the differences between brain states, the REDS technique calculates the target for electrical NIBS by comparing and contrasting MEG, EEG, fMRI, PET, SPECT, ECOG, fNIRS, sMRI, DTI, MRS, and other techniques from two different brain states. In one embodiment, the calculation could be made across individuals where a group of individuals with a desired brain state is compared to a group of individuals with an undesirable brain state; inattentive individuals could be compared to those who are attentive, expert individuals could be compared to novices, depressed individuals could be compared to healthy subjects, brain injured individuals could be compared to healthy individuals, individuals that perform a cognitive operation quickly could be compared to those who work more slowly. This is a “one size fits most” approach to the problem of optimizing electrical NIBS.

In another embodiment, neuroimaging methods compare brain slates within individuals across time, i.e. the brain states associated with correct responses could be compared to those recorded during incorrect responses, attentive could be compared to inattentive, novice could be compared to expert, tired could be compared to wide awake. The comparison of desirable and undesirable brain state within an individual could be used to develop customized electrode arrangements for electrical NIBS in each individual.

In yet another aspect, the present technology can employ various kinds of comparisons of various kinds of brain activities with respect to the same brain in order to determine the most advantageous locations or conformations of electrodes. Thus, the analyses of one or more brain activities that are used to determine the correctly positioned or conformed electrodes and arrays of electrodes for delivering electrical NIBS can include many different parameters. Such parameters include, but are not limited to, the location, amplitude, timing, phase, frequency, and duration of one or more activities in one or more brain areas. The recorded brain activity thus obtained is especially useful when the data recorded gives information about the consistency or causation of amplitude relationships, time relationships, phase relationships, frequency relationships, and the duration relationships across multiple similar events processed by the brain, or across regions in the brain. However, the application of this method to determine the optimal brain targets for electrical NIBS is both innovative and extremely useful. The REDS approach allows both functional (MEG, EEG, fMRI, tNIRS, PET, SPECT, ECOG, and MRS) and structural (sMRI, DTI) comparisons between and within subjects. The idea that electrical NIBS could effect brain structure is novel. van der Merwe et al. (2001) showed that one type of electrical NIBS, TDCS, can alter measures of DTI that indicate the white matter tracts in the brain have decreased radial diffusivity in the hemisphere ipsilateral to stimulation.

This is typically interpreted as increased myelination and/or healthier white matter. This raises many possibilities for the uses of electrical NIBS in rehabilitation and white matter diseases of the brain that occur with aging, Virchow-Robin Perivascular Spaces, deep white matter ischemia, multiple sclerosis, progressive multifocal leukoencephalopathy, post-infections encephalitis, HIV related encephalitis, radiation injury, chemotherapy neurotoxicity (chemobrain), posterior reversible encephalopathy syndrome, central pontine myelinolysis, the leukodystrophies and the adreno leukodystrophies, as well as peripheral and central nervous system damage from traumatic brain injury, concussion, chronic traumatic encephalopathy, spinal cord injury, and stroke. All of these diseases could be treated with the embodiments that do comparisons across or within individuals to identify targets for electrical NIBS in the CNS. The idea of comparing and contrasting brain activity in two conditions or across two populations is not novel. However, evaluating the differences in advanced neuroimaging techniques between populations in order to guide electrical NIBS is quite novel. This approach has been used successfully to double the rate of learning in multiple laboratories and on multiple tasks (Clark et al., 2012; McKinley et al., in press). This allows for evaluation of the differences between desired and undesired brain states.

In another key aspect, users of the present methods, devices and arrays can determine the influence of electrical NIBS to a brain or a group of brains. By way of example, after a target for electrical NIBS is determined in the Recording and Evaluation phases of REDS, the amplitude, polarity, and spatial location of the electrodes that have the greatest influence on the brain structure(s). This is accomplished with finite element modeling. In one aspect, finite element modeling divides the brain, scalp, skull, and surrounding tissues into different layers that can be used to make predictions about the path that electrical NIBS will take through the tissues that surround the brain to get to targeted brain structures. The finite element models are generated from high resolution sMRI. The different gray levels in sMRI images are due to different concentrations of water in the tissues. The different gray levels allow the tissues to be segmented into separate layers and tissue compartments. The layers and tissue compartments are then tessellated across the surface with triangular meshes.

The tessellated meshes can then be assigned a value for how well electricity is conducted through the volume of tissue. Collectively, the layers and tissue volumes in the finite element model are called the forward model. In REDS the area(s) of the brain identified as targets for stimulation to enhance desirable brain states will be virtually activated in the finite element model. The virtual activation of the brain area will project electricity through the forward model and onto the scalp surface. The identified areas of the cortical surface will be the locations for the spatial position of the electrodes. The polarity of the currents that are shown on the scalp will determine the polarity of the currents that are delivered at each spatial position. The strength of the current that is projected onto the scalp surface will determine the proposition of the total current that is delivered at each electrode position.

Stimulation of one or a plurality of brains with electrical NIBS provides manifold advantages and uses. In yet an additional aspect of the present technology, the pattern(s) of stimulation that can be designed to stimulate brain regions that will increase the likelihood of a desired brain state from the Record, Evaluate, and Determine portions of REDS can be programmed into a device of the invention as described herein. Such programming will typically utilize 5 to 10 electrodes on the scalp surface and up to 5 extracephalic electrodes, although any number of electrodes can be adapted to the present devices and methods. The lengths of the up to 10 curved plastic arms that hold the electrodes will be tailored to the position on the scalp that is necessary to target the REDS determined brain structure. The angle at which each aim need to leave the head frame will be set by a mechanism of grooves that locks the arms into the appropriate angle. The polarity and amplitude of electrical NIBS will be set to mimic the pattern observed in the finite element model.

The tailoring of the arm length and the setting of amplitudes and magnitudes can be determined from data collected across individuals or within an individual and for targeting structure or functional differences between desired and undesired brain states. The electrical NIBS device is now programmed to facilitate a one specific desirable brain state or structure. The device would need to be reprogrammed and newly tailored for producing a different desirable brain state. The list of desirable brain states is very large but several specific examples will be given below.

Another useful and innovative with respect to the present technology comprises one or more methods for determining the appropriate target for electrical NIBS in the brain. At present, conventional methods for determining the brain region(s) to be targeted with electrical NIBS are largely based on textbook descriptions of cognitive functions and/or work that details functions that are lost after strokes or brain lesions. Determining target brain tissues with this “lesions and literature” methodology makes multiple fallacious assumptions. These include: 1) all brains are the same, 2) loss of function with lesion indicates the location of function 3) the area of brain directly underneath the electrode is most effected by the electrical NIBS, and 4) laboratory tasks are good proxies for the activities of daily Life in terms of brain activation and prediction of success. The methods disclosed herein in one or more embodiments can be individualized, and customized or matched to appropriate patterns of brain activity, and deployed into daily life to enhance desirable behaviors and reduce undesirable behaviors by electrical NIBS that enhances or reduces activity in appropriate brain regions.

In another embodiment it can be configured in a “one size fits most” configuration that is not designed to be individualized. In both embodiments, the method requires recording brain activity during desirable and undesirable conditions or responses. The patterns of brain activity or structure in the desirable and undesirable conditions are then compared to glean the location and direction in which brain activity or structure must be changed to move from an undesirable to a desirable state of performance. In one embodiment, the effects of electrical NIBS on the brain can then be determined with finite element models that use electrodes in various spatial configurations, strengths, and polarities to determine the most favorable arrangement of a plurality of electrodes that will maximally effect the brain region(s) that is being targeted to alter behavior or deliver therapeutic intervention through excitation or inhibition or structural change.

The optimal spatial configuration, strength, and polarity can then be implemented on the device described above to apply stimulation to the brain to enhance performance or provide therapy through brain excitation, inhibition or structural change. This approach is heretofore referred to, in brief as REDS (Record, Evaluate, Determine, and Stimulate). REDS is distinct from the lesions and literature approach in multiple ways. 1) There are no a priori assumptions made about the location(s), strength(s), or temporal characteristics of brain activity or structure. 2) REDS uses brain states associated with different behavioral patterns in an individual or group of individuals to determine the appropriate brain areas for targeted stimulation. 3) REDS makes no a priori assumptions about how the effects of electrical NIBS are distributed in the brain; it models them virtually. 4) REDS is readily amenable to individualization (iREDS) to not only to specific persons but also to specific brain states within a person as they vary across the day when the embodiment records activity and delivers stimulation as part of a single device.

A third advantageous aspect is that the present methods can be used to target either gray matter (the parts of the brain containing the parts of the neurons that perform the computations necessary for sensation, perception, cognition, emotion, movement, thought, and other behaviors) or the white matter (the connecting tissue between specialized parts of the brain that work together to produce sensation, perception, cognition, emotion, movement, thought, and other behaviors).

According to some preferred specific embodiments of the present technology, an EEG, fMRI or MEG device is used to measure the location, amplitude, and magnitude of time dependent electric and/or magnetic field oscillations that are recorded as one or more outputs or response from the brain in various circumstances. These oscillations that indicate neural activity can be related to normal functions such as the sleep cycle, pattern recognition, learning, teaching, various types of communications and decision making. These signals can also be indicative of abnormal functions caused by sleep deprivation, stress, epilepsy, autism, addiction, and stress disorders.

In one aspect, the time dependent electric field oscillations provide the measurements to one or more devices or networks of the invention, which are able to interpret the measurements, identify signals that are indicative of an abnormal or undesirable function, and generate a modified signal that can be transmitted into the brain using a trans-cranial brain stimulator such as transcranial Direct Current Stimulation (tDCS), transcranial alternating current stimulation (tACS) or Transcranial Magnetic Stimulation (TMS) in order to produce a desired effect. As one example of one specific embodiment of the numerous embodiments of the present invention, if the detected brain output is indicative of the onset of an undesirable brain process, such as the initial stage of a seizure, then the signal generated by the device and delivered to the brain would provide an in-phase, equal magnitude, but opposite sign in order to cancel that signal through destructive interference with the output signal. This superposition of an opposite sign (or cancelling) signal is similar in some aspects to known methods of acoustic noise cancellation commonly used in active acoustic noise cancellation headphones and speakers. Accordingly, a unique feature of the inventions provided in this disclosure is the application of one or more “cancellation signals” within the brain region generating the undesirable output signal.

The application of a pulsed, oscillating, or DC electric field to modify neural activity is known in the art. These approaches typically apply a stimulus in an on/off manner based on a prescribed dose/time relationship. In stark contrast, according to various embodiments, the presently described invention utilizes closed loop feedback in order to provide active modification of a device-generated input signal in response to the brain's output signal.

According to another embodiment of the invention, a feedback device is connected to electrodes that are place on the head in locations that are optimized for activation or deactivation of signals of interest that are produced by the brain. For instance, if the output is indicative of the early stage of a seizure in a localized brain region, the electrodes are located to provide or direct a cancellation wave to the part of the brain responsible for generating the early stage seizure related signals in order to prevent the growth of wide spread coupled brain oscillations. According to various embodiments, at least a portion of the feedback device could take the form of a headset, cap, hat, helmet, head draping, headband or pillow.

According to yet another embodiment, the feedback device could be placed and optimized to encourage the brain to generate particular signals, or cycles of particular signals, that are adapted and arranged to fulfill one or more desired functions. For example, a suitable application envisaged by the inventor is to treat sleep deprivation caused by undesirable rapid transition from non-REM sleep into REM sleep. In this one of many embodiments, the purpose of the input field would be to entrain the signals produced by the brain that are associated with healthy sleep cycles and reduce the frequency of maladaptive patterns of sleep. In one alternative, the feedback device could be designed to encourage restorative slow wave sleep and prevent quick or premature transition into REM sleep. Control of sleep brain patterns, either by preventing undesirable signals or by controlling the signal patterns over time could help reduce or prevent nightmares, and/or produce sleep that is more restorative over shorter durations, essentially allowing for an electrically stimulated powernap. Similar patterns could be profoundly useful for treatment of disorders such as post-traumatic stress disorder (PTSD).

Alternatively stated, the feedback device could be designed such that one or more electrodes are placed so as to direct the feedback device-generated signal towards those regions of the brain (the target regions. portions or structures) that are responsible for generating the signal of interest. In some embodiments, arrays of electrodes may be utilized to localize or concentrate feedback device-generated signals to one or more specific regions (the target regions, portions or structures) of the brain.

According to still another embodiment, the feedback device could be placed and optimized not to cancel an undesirable signal, but rather to amplify a desirable, naturally occurring signal. In yet another alternative of some of the key present methods, the feedback device could cancel or suppress some signals of interest, while amplifying others. For example, in applications (methods) to enhance memory, learning, or pattern recognition, the detection of a desirable signal would allow the feedback device to amplify that signal associated with storage of the information of interest separately, or in concert, with suppressing cognitive processes that compete for resources that could be used to encode memory.

For example, in one embodiment adapted for the purpose of teaching one or more languages, the brain activity and structure of one or more subject groups are recorded with one or more neuroimaging methods with respect to both desirable and undesirable brain states defined as fluent and non-fluent, respectively, according to the present methods. The differences between the desirable and undesirable states are then evaluated in order to produce an electrode array that will facilitate language learning in many individuals.

As an aspect of teaching languages, individual enhancement strategies can be tailored, developed or customized to one or a group of people. As an example of certain parameters of methods of the invention, the brain activity and structure-of a single subject can be recorded in desirable and undesirable brain states. The data thereby obtained can be used, for example, to determine and teach such nuances of language learning such as inflection, accent and rhythm. The difference between such desirable and undesirable brain states can advantageously be evaluated to produce an electrode array that is customized to enhance performance in a particular individual and may or may not be applicable to other individuals.

Similar strategies can be applied to many different types of learning dynamics. Thus, general aspects of the present methods can be applied to thereby achieve numerous different learning scenarios. As additional examples, the present methods, techniques and procedures, with the benefit of the present specification, can be directed toward the reduction of fatigue, of either an individual or a group.

According to yet another embodiment, two or more feedback device could be in electrical communication with one another. In such embodiments, a feedback device of a first individual could transmit information to the feedback device of another individual or to the feedback devices of a group of individuals in order to enhance team performance by manipulating attention, engagement, and/or coordination of the group.

In one of many possible military applications involved in a small group attempting to deal with ambiguous unstructured information, the coupling of multiple feedback devices would lead to enhanced detection of relevant information and coordination of the group. For example, if the feedback device of one member of a group identified brain waves associated with increased alertness, for example in response to the individual noticing “unusual or suspicious activity,” the feedback devices of the other members of the group could be programmed to increase alertness for all members of the group within a predetermined proximity, or those who are chosen to be in a particular communications network.

According to another embodiment, the individual feedback device could be coupled to remotely located computers to provide additional real time processing and memory for each of the feedback devices. These computers could then be connected into a feedback and control system to provide overall management and coordination of the ensemble. For example, the feedback devices could be used to enhance the performance of a team of cyber defenders who are dealing with rapidly changing ambiguous information. The ability to detect pre-conscious patterns is known in the art, and the sharing of these preconscious detections would enhance the speed and coordination of the group. The ability to amplify this detection capability of the individuals and the group would lead to substantial performance enhancements of both the individuals, and of the group as a whole. According to yet another embodiment, rather than generating a signal that is equal and opposite to the signal of interest, the feedback device could introduce white noise so as to disrupt the signal of interest.

In accordance with the several objects of the invention, a variably configurable electrode array is provided, wherein the array comprises: A) at least two electrodes, wherein each of the electrodes is operationally connected to the other electrodes, or to at least one microprocessor; B) a housing adapted and arranged for variably positioning the electrodes with respect to one another, and for variably positioning each of the electrodes respectively in operational proximity to one or more regions, areas or points of a scalp of a subject upon which the array is placed; C) at least one microprocessor located in operational proximity to the housing, wherein the microprocessor is adapted and arranged to process data collected by means of the electrodes; and D) at least one data storage module located in operational proximity to the array, wherein the module is adapted and arranged to be operationally connectable to one or more of the at least two electrodes and the at least one microprocessor; and E) at least one software means suitable for storing software, wherein the software means is adapted and arranged for one or more of a) operating one or more functions of the array, b) storing data collected by the array and c) processing data. Preferably, an array of the invention further comprises a battery or other means adapted and arranged for providing electrical power to the array or to objects or modules attached to the array.

In one aspect, the present technology can employ various kinds of comparisons of various kinds of brain activities with respect to the same brain in order to determine the most advantageous locations or conformations of electrodes. Thus, the analyses of one or more brain activities that are used to determine the correctly positioned or conformed electrodes and arrays of electrodes for delivering electrical NIBS can include many different parameters. Such parameters included, but are not limited to, the location, amplitude, timing, phase, frequency, and duration of one or more activities in one or more brain areas. The recorded brain activity thus obtained is especially useful when the data recorded gives information about the consistency or causation of amplitude relationships, time relationships, phase relationships, frequency relationships, and the duration relationships across multiple similar events processed by the brain, or across regions in the brain. In yet another series of embodiments of the invention, one or more kits are provided. In some preferred embodiments, a kit of the invention comprises A) at least one array according, to claim 1, B) software means necessary to operate the array in all desired aspects, and C) task software contained in operational connection or within the array housing directed toward one or more specific purposes. Task software in this context can be any software adapted and arranged for facilitating any task for which the kit is directed. Task software for use with the invention is preferably one or more from the group comprising language learning software, ability testing software, diagnostic software, and intervention software.

In yet another set of embodiments, the present invention includes one or more networks, wherein each network comprises a plurality of variably configurable electrode arrays as defined in claim 1, and wherein the plurality of arrays are adapted and arranged to be in operative communication with another while one or a plurality of the arrays are in operational proximity to one or a plurality of the scalps of one or more subjects. A network of the invention may further comprise a control module, wherein the control module is adapted and arranged for facilitating a plurality of control functions of the arrays and of the network. Preferable control functions of the network include, as examples, one or more of oscillations of a particular frequency, time varying functions on a single electrode and coordinated with time varying functions on a plurality of electrodes that can vary with respect to correlation, causality, duration, phase, latency, amplitude, and frequency.

In some preferred embodiments of the invention, one or more of the microprocessor, the software means, and the data storage module are one or more of i) in operative communication with one another, ii) in operative communication with one or more networks, and iii) in operative communication with humans or computer systems external to the array. In a somewhat similar context, or more of the microprocessor, the software means, and the data storage module are in telemetric or other communication with a computerized network, or with one or more other means for doing one or more of a) recording data obtained or contained in connection with the array, b) operating the array, and c) storing data contained or obtained in connection with operation of the array.

In one preferred embodiment, a kit of a device of the invention may comprise a clamshell -type housing adapted and arranged to contain a plurality, such as inside 6, 8 or 10, electrodes, one or more preloaded gel packs adapted for facilitating all effective interface between the electrodes and the skin of the scalp, as well as electrodes, electrode holders, and a head frame. As another advantageous aspect of some preferred embodiments of the invention, the housing is provided with one or more means for operatively and reversibly containing one or more of the microprocessor, the data storage module and the software means such that substitute or interchangeable microprocessors, storage modules and software means in operative communication with the array can be exchanged, replaced or substituted when desired. Thus, one or more arrays of the invention can be put to a myriad of selected uses.

In one advantageous set of aspects, many different types of software can be used to direct or control the various functions, operational parameters, and characteristics of the invention, including the following, which are provided as examples, and not as limitations of the functions or uses of the invention. Thus, software for use with or in the invention may comprise one or more of software means for setting the stimulation duration across all electrodes of the array; software means for setting the stimulation intensity at each electrode; software means for setting the stimulation polarity at each electrode; software means for setting the stimulation DC offsets at each electrode; software means for setting the time varying function at each electrode; software means for setting the ramp up and ramp down times at each electrode; software means for checking the impedance at each electrode; and software means for monitoring the impedance at each electrode: software means for controlling safety override voltages at each electrode; software means for setting the lockout time period across all electrodes; software means for effecting one or more electrode maintenance routines across all electrodes; software means for checking one or more battery parameters before stimulation begins; software means for locking the settings to prevent tampering with the software and certain settings of the device; software means for operatively communicating with the array software interface for setting stimulation parameters; software means for operatively connecting a plurality of arrays to one another, and software means for performing finite element modeling.

Additional software means includes one or more software means for one or more of determining and redetermining the optimal spatial location of one or more electrodes with respect to the scalp and with respect to the housing, software means for one or more of determining and redetermining the optimal polarity of each electrode at each location, software means for one or more of determining and redetermining the intensity of the current delivery at each electrode location, software means for one or more of determining and redetermining the time varying wave form with respect to each electrode; software means for generating time varying functions that mimic one or more brain activities, software means for detecting EEG signals, software means for generating feedback to alter one or more electrical or structural brain activities, software means for interpreting and classifying detected EEG activity as a desired or an undesirable brain state, and software means for providing feedback to the subject in the form of one or more types of electrical brain stimulation, as well as software means for one or more of determining and redetermining one or more tasks of the arrays.

As yet another advantageous characteristic of certain preferred embodiments of the invention, one or more of the microprocessor, the data storage module and the software means can be reversibly provided in operation proximity to one or more electrodes of the array. Thus, arrays of the invention can be programmed and reprogrammed by switching out various physical and software components. In addition, one or more of the microprocessor, the data storage module and the software means can be permanently provided in operation proximity to one or more electrodes of the array.

As yet other advantages of preferred embodiments of the invention, the array of one or more of the housing and electrodes can be adapted and arranged for determining the respective optimal operational parameters and configurations of one or more external scalp electrodes, pluralities of electrodes or electrode arrays with respect to a single subject brain. Moreover, one or more of the microprocessor, the data storage module and the software means can be adapted and arranged to be reconfigured during operation of the array on a subject. Thus, as arrays of the invention are operating and communicating with various systems, their various functions can be changed or redirected as wanted or needed.

As additional adaptive advantages and characteristics of some preferred embodiments of the present invention, the present arrays can be provided in self-contained embodiments, or kits. Although many preferred embodiments of self-contained arrays are within the spirit and scope of the invention, typical embodiments include those that are provided as kits. Thus, a typical kit embodiment of the present variably configurable electrode array would include A) at least one array, wherein the array comprises: A) at least two electrodes, wherein each of the electrodes is operationally connected to the other electrodes, or to at least one microprocessor; B) a housing adapted and arranged for variably positioning the electrodes with respect to one another, and for variably positioning each of the electrodes respectively in operational proximity to one or more regions, areas or points of a scalp of a subject upon which the array is placed; C) at least one microprocessor located in operational proximity to the housing, wherein the microprocessor is adapted and arranged to process data collected by means of the electrodes; and D) at least one data storage module located in operational proximity to the array, wherein the module is adapted and arranged to be operationally connectable to one or more of the at least two electrodes and the at least one microprocessor; and E) at least one software means suitable for storing software, wherein the software means is adapted and arranged for one or more of a) operating one or more functions of the array, b) storing data collected by the array and c) processing data. It may also include software means necessary to operate the array in all desired aspects, and d) task software contained in operational connection or within the array housing directed toward one or more specific purposes.

A self-contained embodiment of the invention may also include wherein the task software is one or more from the group comprising language learning software, ability testing software, diagnostic software, and any other software means adapted and arranged for effecting one or more diagnostic, evaluational teaching, and redirective tasks.

As yet another positive aspect, embodiments of the invention include also where pluralities of electrodes or pluralities of arrays are networked plurality of variably configurable electrode arrays as described herein, wherein the plurality of arrays are adapted and arranged to be in operative communication with one another while one or a plurality of the arrays are in operational proximity to one or a plurality of the scalps of one or more subjects. As in certain other embodiments of the present invention, networked pluralities of arrays of the invention can be adapted and arranged to measure, test, evaluate, teach, redirect, record and assess numerous abilities, characteristics, values, capabilities and aspects of one or more brains.

In accordance with the several objects of the invention, a variably configurable electrode array is provided in the context of effecting various methods, wherein one of the methods is a method for determining the optimum operational parameters of one or more neurological electrodes or electrode arrays, the method comprising the steps or actions of: A) recording one or more brain activities of a subject to obtain one or more brain electrical activity patterns of the subject brain during i) one or more desirable brain states, and ii) one or more undesirable brain states, to thereby obtain datasets with respect to each activity, wherein the datasets with respect to the desirable brain states and the undesirable brain states datasets are correlated; and then B) evaluating any of the correlated datasets or patterns obtained with respect to corresponding desirable and undesirable brain states to obtain one or more difference datasets; and then C) utilizing the difference datasets to effect a determination of one or more target regions, portions or locations of the subject brain. The present method may also comprise further the step or action of D) effecting stimulation of the determined target regions, portions or locations of the target brain with electrical NIBS to the extent necessary to effect desired changes in the brain patterns or activities.

As another advantage of the present method, one or more of the polarity, intensity, and spatial distribution of the effected stimulation can be utilized to produce a desired excitation or inhibition of one or more of the regions, portions or locations of the target brain. Moreover, the stimulation is adapted and arranged to effect the maximal desired influence at the one or more regions, portions or locations of the target brain such that changes in the brain effected by the NIBS influences the targets to move from one or more undesirable states to one or more desirable states.

In another significant aspect of many preferred embodiments of methods of the present technology, finite element modeling is used to filter or refine the datasets and images obtained by electrodes and arrays of the invention. As examples, finite element modeling of one or more of the target brain, scalp, skull and associated tissues is utilized in order to determine the most advantageous parameters of the various possible configurations and variations of the present electrodes and arrays.

Method of the present technology may also utilize wherein the brain activities are recorded by one or more of magnetoencephalography (MEG), electroencephalography (EEG), functional magnetic resonance imaging (fMRI), positron emission tomography (PET). single photon emission computed tomography (SPECT), electrocorticography (ECOG), structural magnetic resonance imaging (sMRI), diffusion tensor imaging (DTI), magnetic resonance spectroscopy (MRS), and/or functional near infrared spectroscopy (fNIRS) during desirable and non-desirable brain states, i.e. expert vs. novice, highly attentive vs. non-attentive, awake vs. fatigued, correct responses vs. incorrect responses, injured vs. uninjured.

In accordance with the many objects of the present invention, methods for determining the optimal operational parameters and configurations of one or more external scalp electrodes, pluralities of electrodes or electrode arrays with respect to a single subject brain, are provided. In one significant embodiment, the method comprises the steps or actions of: A) operating one or more of the electrodes or electrode arrays to create one or more recordings of activities, states, or structures of a subject brain to obtain data with respect to the one or more activities, states, or structures of the subject brain during one or more desirable brain states, and one or more undesirable brain states, to thereby collect obtained datasets regarding each activity, state or structure with respect to the desirable brain states and with respect to the undesirable brain states, wherein the obtained datasets are adaptable to one or more comparisons: then B) effecting one or a plurality of comparisons of the obtained datasets with respect to corresponding desirable and undesirable brain states to obtain one or more difference datasets; then C) evaluating the difference datasets to effect a determination of one or more target regions, portions or locations of the subject brain. The present method may also include the further step of D) utilizing the obtained datasets and the difference datasets to effect one or more redesigns or reconfigurations of the one or more external electrodes or electrode arrays to arrive at an improved or optimized electrode or electrode array.

As examples of the variability and adaptability of the present methods, electrodes and arrays, the one or more redesigns or reconfigurations can be made with respect to many factors, functions and uses. These include, as examples, one or more of the three-dimensional relationships between or among the electrodes, pluralities of electrodes or electrode arrays, the three-dimensional relationships between or among the electrodes, pluralities of electrodes or electrode arrays, one or more electrode carriers or frames, and the scalp upon which the electrodes, pluralities of electrodes or electrode arrays are placed.

This is also true with respect to those situations wherein the adaptations, redesigns or reconfigurations of the electrodes, pluralities of electrodes or electrode arrays are made with respect to one or more of the difference in images or patterns obtained, for example, as part of the present methods and arrays, finite element modeling in which the brain regions, portions, locations, or structures are selected in a brain virtualized in a finite element model can be used as a filter to determine the locations on the scalp where electrodes would be most effective in delivering current to the identified brain area. Examples of parameters that can be indicated by the finite element modeling include, as examples, the spatial location of electrodes on the scalp, the polarity of the electrode at each location, the intensity of the current delivery at each electrode location, and the time varying wave form at each electrode location.

The present method may also comprise the step or action of E) utilizing the improved or optimized electrode, plurality of electrodes or electrode array to stimulate the target brain to thereby test the design of the improved electrode or electrode array and to obtain additional datasets; as well as the step of F) utilizing the additional datasets to further redesign or reconfigure the improved electrode or plurality of electrodes in an array to arrive at a final electrode or plurality of electrodes in an array.

In accordance with yet additional positive aspects and adaptations of the present technology, one of the present methods includes that for determining the optimal operational parameters and configurations of one or more external scalp electrodes with respect to a plurality of subject brains, the method comprising the steps or actions of A) creating one or more records of one or more brain activities, states, or structures with respect to the plurality of the subject brains to obtain data with respect to one or more brain electrical activities of the plurality of the subject brains during i) one or more desirable brain states, and ii) one or more undesirable brain states, to thereby collect obtained datasets regarding each activity, state, or structure with respect to the desirable brain states and with respect to the undesirable brain states, wherein the obtained datasets are adaptable to one or more comparisons; then B) effecting one or a plurality of comparisons of the obtained datasets with respect to corresponding desirable and undesirable brain states of the plurality of brains to obtain one or more difference datasets; and then C) evaluating the difference datasets to effect a determination of one or more target regions, portions or locations of the plurality of subject brains.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the light to physically incorporate into this specification any and all materials and information from any such cited patents or publications. The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth.

Under no circumstances may the patent be interpreted to be limited to the specific examples or embodiments or methods specifically disclosed herein. The terms and expressions that have been employed are used as terms of description and not of limitation, and there is no intent in the use of such terms and express ions to exclude any equivalent of the features shown and described or portions thereof, but it is recognized that various modifications are possible within the scope of the invention as claimed. Thus, it will be understood that although the present invention has been specifically disclosed by preferred embodiments and optional fatures, modification and variation of the concepts herein disclosed may be resorted to by those skilled in the art, and that such modifications and variations are considered to be within the Scope of this invention as defined by the appended claims.

The invention is described broadly and generically herein, while also providing descriptions Figures, photo-images and diagrams of various specific or exemplary embodiments of elements or portions of the invention. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

What is claimed is:

1. A variably configurable electrode array, comprising:
A) at least two electrodes, wherein each of the electrodes is operationally connected to the other electrodes, or to at least one microprocessor;
B) a housing adapted and arranged for variably positioning the electrodes with respect to one another, and for variably positioning each of the electrodes respectively in operational proximity to one or more regions, areas or points of a scalp of a subject upon which the array is placed;
C) at least one microprocessor located in operational proximity to the housing, wherein the microprocessor is adapted and arranged to process data collected by means of the electrodes; and
D) at least one data storage module located in operational proximity to the array, wherein the module is adapted and arranged to be operationally connectable to one or more of the at least two electrodes and the al least one microprocessor; and
E) at least one software means suitable for storing software, wherein the software means is adapted and arranged for one or more of a) operating one or more functions of the array, b) storing data collected by the array and c) processing data.

2. The array of claim 1, further comprising a battery or other means adapted and arranged for providing electrical power to the array or to objects or modules attached to the array.

3. The array of claim 1, wherein one or more of the microprocessor, the software means, and the data storage module are one or more of i) in operative communication with one another, ii) In operative communication with one or more networks, and iii) in operative communication with humans or computer systems external to the array.

4. The array of claim 1, wherein one or more of the microprocessor, the software means, and the data storage module are in telemetric or other communication with a computerized network, or with one or more other means for doing one or more of a) recording data obtained or contained in connection with the array, b) operating the array, and c) storing data contained or obtained in connection with operation of the array.

5. The array of claim 1, wherein the housing is provided with one or more means for operatively and reversibly containing one or more of the microprocessor, the data storage module and the software means such that substitute or interchangeable microprocessors, storage modules and software means in operative communication with the array can be exchanged, replaced or substituted when desired.

6. The array of claim 1, further comprising a head frame adapted and arranged for holding one or more of the electrodes and the housing in one or more positions with respect to a scalp upon which the electrodes or housing are positioned.

7. The array of claim 1, wherein the software means comprises one or more of software means for setting the stimulation duration across all electrodes of the array;
software means for setting, the stimulation intensity at each electrode;
software means for setting the stimulation polarity at each electrode;
software means for setting the stimulation DC offsets at each electrode;
software means for setting the time varying function at each electrode;
software means for setting the ramp up and ramp down times at each electrode;
software means for checking the impedance at each electrode; and
software means for monitoring the impedance at each electrode.

8. The array of claim 1, wherein the software means comprises:
one or more of software means for controlling safety override voltages at each electrode;
software means for setting the lockout time period across all electrodes,
software means for effecting one or more electrode maintenance routines across all electrodes,
software means for checking one or more battery parameters before stimulation begins,
software means for locking the settings to prevent tampering with the software and certain settings of the device,
software means for operatively communicating with the array soft are interface for setting stimulation parameters,
software means for operatively connecting a plurality of arrays to one another,
software means for performing finite element modeling,
software means for one or more of determining and redetermining the optimal spatial location of one or more electrodes with respect to the scalp and with respect to the housing,
software means for one or more of determining and redetermining, the optimal polarity of each electrode at each location,
software means for one or more of determining and redetermining the intensity of the current delivery at each electrode location,
software means for one or more of determining and redetermining the time varying wave form with respect to each electrode,
software means for generating time varying functions that mimic one or more brain activities,
software means for detecting EEG signals,
software means for generating feedback to alter one or more electrical or structural brain activities,
software means for interpreting and classifying detected EEG activity as a desired or an undesirable brain state,
software means for providing feedback to the subject in the form of one or more types of electrical brain stimulation.

9. array of claim 5, wherein one or more of the software means comprises means for one or more of determining and redetermining one or more tasks of the array.

10. The array of claim 1, wherein one or more of the microprocessor, the data storage module and the software means are reversibly provided in operation proximity to one or more electrodes of the array.

11. The array of claim 1, wherein one or more of the microprocessor, the data storage module and the software means are permanently provided in operator proximity to one or more electrodes of the array.

12. The array of claim 1, wherein the housing and electrodes are adapted and arranged for determining the respective optimal operational parameters and configurations of one or more external scalp electrodes, pluralities of electrodes or electrode arrays with respect to a single subject brain.

13. The array of claim 1, wherein one or more of the microprocessor, the data storage module and the software means are adapted and arranged to be reconfigured during operation of the array on a subject.

14. A kit comprising A) at least one array according to claim 1, B) software means necessary to operate the array in all desired aspects, and C) task software contained in operational connection or within the array housing directed toward one or more specific purposes.

15. The kit of claim 14, wherein the task software is one or more from the group comprising language learning software, ability testing software, diagnostic software, and intervention software.

16. The kit of claim 14, wherein the software means are one or more selected from the group comprising:
software means for setting the stimulation duration across all electrodes of the array;
software means for setting the stimulation intensity at each electrode;
software means for setting the stimulation polarity at each electrode;
software means for setting the stimulation DC offsets at each electrode:
software means for setting the time varying function at each electrode;
software means for setting the ramp up and ramp down limes at each electrode;
software means for checking the impedance at each electrode; and
software means for monitoring the impedance at each electrode;
software means for controlling safety override voltages at each electrode;
software means for setting the lockout time period across all electrodes;
software means for effecting one or more electrode maintenance routines across all electrodes;
software means for checking one or more battery parameters before stimulation begins;
software means for locking the settings to prevent tampering with the software and certain settings of the device;
software means for operatively communicating with the array software interface for setting stimulation parameters;
software means for operatively connecting, a plurality of arrays to one another, and
software means for performing finite element modeling.

17. The kit of claim 14, wherein the software means are one or more selected from the group comprising:
software means for one or more of determining and redetermining the optimal spatial location of one or more electrodes with respect to the scalp and with respect to the housing.
software means for one or more of determining and redetermining the optimal polarity of each electrode at each location,
software means for one or more of determining and redetermining the intensity of the current delivery at each electrode location,
software means for one or more of determining and redetermining the time varying wave form with respect to each electrode;
software means for generating time varying functions that mimic one or more brain activities,
software means for detecting EEG signals,
software means for generating feedback to alter one or more electrical or structural brain activities,
software means for interpreting, and classifying detected EEG activity as a desired or an undesirable brain state, and
software means for providing feedback to the subject in the form of one or more types of electrical brain stimulation, as well as
software means for one or more of determining and redetermining one or more tasks of the arrays.

18. A network comprising a plurality of variably configurable electrode arrays as defined in claim 1, wherein the plurality of arrays are adapted and arranged to be in operative communication with another while one or a plurality of the arrays are in operational proximity to one or a plurality of the scalps of one or more subjects.

19. The network of claim 18, further comprising a control module, wherein the control module is adapted and arranged for facilitating a plurality of control functions of the arrays and of the network.

20. The network of claim 19, wherein the control functions are one or more of oscillations of a particular frequency, time varying functions on a single electrode and coordinated with time varying functions on a plurality of electrodes that can vary with respect to correlation, causality, duration, phase, latency, amplitude, and frequency.

21. The kit of claim 14, wherein the kit is both self-contained and adapted and arranged to be in wireless operational communication with one or more other devices.
